(19) United States
(12) Patent Application Publication
(10) Pub. No.: US 2026/0321419 A1
(43) Pub. Date: Oct. 15, 2026

(54) HEALTH ABNORMALITY DETECTION SYSTEM, METHOD AND COMPUTER READABLE STORAGE MEDIUM

(71) Applicant: WellnessMind Al Ltd, Anaheim, CA (US)

(72) Inventors: Xiaozhong Liu, Worcester, MA (US); Zhuoren Jiang, Hangzhou (CN)

(21) Appl. No.: 19/178,347

(22) Filed: Apr. 14, 2025

(51) Int. Cl. G16H 50/20; G06F 40/40; G16H 10/60; G16H 20/10; G16H 40/67; G16H 50/30

(52) U.S. Cl. CPC G16H 50/20; G16H 10/60; G16H 20/10; G16H 40/67; G16H 50/30; G06F 40/40

(57) ABSTRACT

System, method, and computer-readable storage medium for health abnormality detection are described, which enable dynamic interaction of multi-layer intelligent memories, comprising a multi-modal physiological data collection module, an initial abnormality detection module, a multi-layer intelligent memory coordination module and a generative interaction and feedback module.

### TECHNICAL FIELD

The present disclosure generally relates to a health abnormality detection system, method and computer readable storage medium, and more particularly to a health abnormality detection system, method and computer readable storage medium that enables dynamic interaction of multi-layer intelligent memories.

### BACKGROUND

Although existing wearable devices can collect basic physiological data such as heart rate and blood oxygen, the health abnormality detection function thereof has certain limitations, mainly in the following aspects:
(1) lack of multi-layer memory support: the current system is unable to effectively store user's long-term health data, making it difficult to combine with real-time monitored information for comprehensive assessment, which is particularly inadequate in chronic disease management. A single memory mechanism such as short-term data caching can lead to a lack of dynamic adaptability of the system to historical data;
(2) limited dynamic interaction capability: the current system mostly generate interaction questions with fixed inquiry templates or rules, making it difficult to be dynamically adjusted according to the user's personalized needs, and are unable to make full use of real-time feedback to optimize the detection and diagnosis process;
(3) insufficient integration of physiological data and user health records: the current system analyzes anomalies based only on current physiological data, ignoring the user's historical health information and behavioral patterns.

Therefore, there is a need in the prior art for a health abnormality detection system that enables multi-layer memory support, dynamic interaction capabilities and better integration of physiological data and user health records.

### SUMMARY

The disclosure relates to a health abnormality detection system, method and computer readable storage medium that can realize multi-layer intelligent memory dynamic interaction, which can realize wearable device collecting multi-modal physiological signals and combining multi-layer intelligent memory coordination and dynamic interaction.

This disclosure relates to a health abnormality detection system, that enables dynamic interaction of multi-layer intelligent memories, comprising a multi-modal physiological data collection module, an initial abnormality detection module, a multi-layer intelligent memory coordination module and a generative interaction and feedback module; wherein, the multi-modal physiological data collection module collects user's multi-modal physiological signals in real time through wearable devices, preprocesses the collected data and generates time-series data, the generated time-series data are provided as input data for subsequent abnormality detection, diagnosis and treatment; the initial abnormality detection module performs initial abnormality detection on the time-series data generated by the multi-modal physiological data collection module, and classifies abnormality detection results; the multi-layer intelligent memory coordination module manages the user's quick memory, deep memory and immediate memory, and performs cooperative optimization of health data through memory retrieval and dynamic update; wherein, the quick memory comprises recent health fluctuations, the deep memory stores the historical health data of the user, the immediate memory dynamically integrates the quick memory and the deep memory and serves as a dynamic input to provide support for abnormality detection and personalized inquiry; the generative interaction and feedback module combines abnormality detection results, user's health memory and medical knowledge base, dynamically generates personalized consultation questions through the generative large language model to interact with the user; simultaneously, the user feedback is embedded and processed for dynamic updating of memory.

In some embodiment, the initial abnormality detection module performs multi-modal embedding generation and classification, dynamic threshold adjustment and abnormality classification, in which, the multi-modal embedding generation and classification is based on a deep sequence model to accept the input time-series data of the physiological signals and convert into a multi-modal health signal embedding vector through an encoder; the dynamic threshold adjustment adopts an adaptive weight mechanism to dynamically adjust an abnormality detection threshold of each signal according to real-time data; the abnormality classification is based on a classifier of the embedding vector to output abnormality category and emergency degree, the results of which are transmitted to a memory module for dynamic update and used as input for generative interaction and feedback module to optimize a personalized consultation process.

Preferably, the formula for dynamic threshold adjustment is as follows:

Tᵢ=T₀+α·Δxᵢ
where, Tᵢ is an adjusted threshold, T₀ is an initial threshold, α is an regulatory factor, Δxᵢ is a degree to which the signal deviates from the mean.

In some embodiment, a classifier based on the embedding vector E outputs the abnormality category and the emergency degree, a classification probability distribution of which is generated by the following formula:

P(y|E)=f_{c}(W·E+b)
where, y represents different abnormality category, f_{c} represents the distribution function of the probability classification generated by the classifier, W and b represent the weight matrix and bias vector of the classifier respectively, the system outputs the abnormality category y and emergency severity s_{E} based on P(y|E) and generates text explanation, which is passed to the generative interaction and feedback module.

Preferably, in the multi-layer intelligent memory coordination module, the system uses a memory retrieval model to match physiological signal embedding vector, quick memory embedding vector and deep memory embedding vector, and uses attention mechanism to calculate correlation thereof.

Preferably, the dynamic updating of the memory includes quick memory updating process and deep memory expansion process; in the quick memory updating process, the system is based on a sliding time window, and combines with a feedback data to generate a new quick memory embedding vector and replace expired information; in the deep memory expansion process, based on access frequency, information relevance and migration rules, the quick memory information is dynamically transferred and integrated into the deep memory.

Preferably, in the generative interaction and feedback module, the system retrieves domain knowledge related to abnormality detection results from the medical knowledge base, by combing the abnormality category, health signal and user related health memory data.

Preferably, the generative interaction and feedback module combines a relevant health memory, an initially detected abnormality category and an initially detected emergency severity, an embedding vector of multi-modal health signals and a health domain knowledge, and the generative large language model dynamically generates the question sequence, the system interacts with the user through natural language questions.

Another respect of this disclosure relates to a health abnormality detection method, that enables dynamic interaction of multi-layer intelligent memories, comprising following steps: collecting user's multi-modal physiological signals in real time through wearable devices, preprocessing the collected data and generating time-series data; performing initial abnormality detection on the generated time-series data, and classifying abnormality detection results; managing user's quick memory, deep memory and immediate memory, and performing cooperative optimization of health data through memory retrieval and dynamic update; wherein, the quick memory comprises recent health fluctuations, the deep memory stores historical health data of the user, the immediate memory dynamically integrates the quick memory and the deep memory and serves as a dynamic input to provide support for abnormality detection and personalized inquiry; combining abnormality detection results, user's health memory and medical knowledge base, dynamically generating personalized consultation questions through the generative large language model to interact with the user; wherein, user's feedback is embedded for dynamic updating of memory.

In some embodiment, performing initial abnormality detection comprises the following steps: performing multi-modal embedding generation and classification process, in which, based on the deep sequence model, accepting the input time-series data of physiological signals and converting into a multi-modal health signal embedding vector through an encoder; performing dynamic threshold adjustment process, in which, adopting an adaptive weight mechanism to dynamically adjust the abnormality detection threshold of each signal according to real-time data; and performing abnormality classification process, in which, based on a classifier of the embedding vector, outputting abnormality category and emergency degree, the results of which are transmitted to a memory module for dynamic update and used as input for optimizing a personalized consultation.

Preferably, in the process of performing cooperative optimization of health data through memory retrieval and dynamic update, the method uses a memory retrieval model to match physiological signal embedding vector, quick memory embedding vector and deep memory embedding vector, and uses attention mechanism to calculate correlation thereof.

Preferably, the dynamic updating of the memory includes quick memory updating process and deep memory expansion process; in the quick memory updating process, the method is based on a sliding time window, and combines with feedback data to generate a new quick memory embedding vector and replace expired information; in the deep memory expansion process, based on access frequency, information relevance and migration rules, the quick memory information is dynamically transferred and integrated into the deep memory.

In some embodiment, further comprising retrieving domain knowledge related to abnormality detection results from the medical knowledge base, by combing the abnormality category, health signal and user related health memory data.

In some embodiment, further comprises the following steps: combing a relevant health memory, an initially detected abnormality category and an initially detected emergency severity, an embedding vector of multi-modal health signal and health domain knowledge; dynamically generating a question sequence, and interacting with the user through natural language questions.

Yet another respect of this disclosure relates to a computer readable storage medium storing a program that enables a computer to execute the health abnormality detection method as described above.

This disclosure innovatively designs a dynamic management model of multi-layer intelligent memory by combining the user's electronic health record. By combining the real-time data acquisition of wearable devices and the dynamic interaction of multi-layer intelligent memory, it can provide accurate, real-time and personalized solutions for the detection of health abnormalities.

### BRIEF DESCRIPTION OF THE DRAWINGS

FIG. 1 is a schematic framework of the health abnormality detection system described in this disclosure.

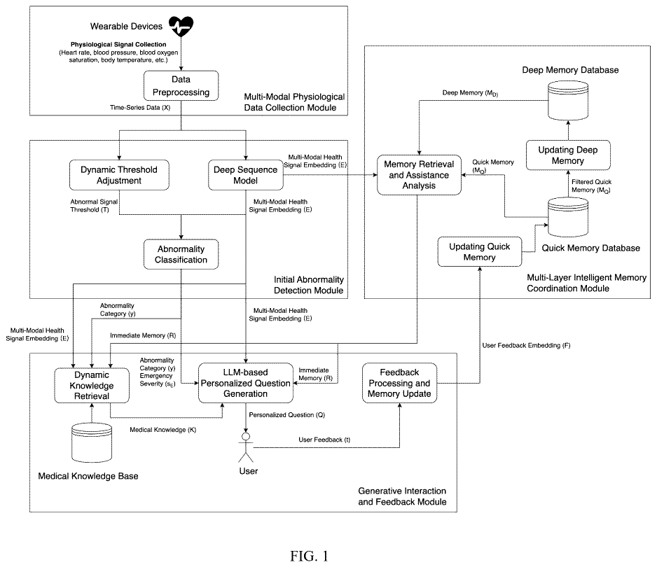

### DETAILED DESCRIPTION

In order to make the objectives, technical solutions and advantages of the present disclosure clearer, the embodiments of the present disclosure will be described below in detail with reference to the accompanying drawings. The technical features in different embodiments of the present disclosure may be combined with each other if not conflicted.

The present disclosure provides a health abnormality detection system that enables dynamic interaction of multi-layer intelligent memories, including a multi-modal physiological data collection module, an initial abnormality detection module, a multi-layer intelligent memory coordination module, and a generative interaction and feedback module. The multi-modal physiological data collection module is used to collect user's multi-modal physiological signals in real time through wearable devices, preprocess the collected data, generate time-series data, and provide input for subsequent abnormality detection, diagnosis and treatment. The initial abnormality detection module performs initial abnormality detection on the time-series data generated by the multi-modal physiological data collection module, and classifies abnormality detection results. The multi-layer intelligent memory coordination module is used to manage the user's quick memory, deep memory and immediate memory, and to perform cooperative optimization of health data through memory retrieval and dynamic update. Wherein, quick memory captures recent health fluctuations, deep memory stores user's historical health data, and immediate memory dynamically integrates quick memory and deep memory to provide support for abnormality detection and personalized consultation. The generative interaction and feedback module combines abnormality detection results, user health memory and medical knowledge base, dynamically generates personalized consultation questions through a generative large language model to interact with users; and embeds the user feedback for updating the quick memory or migrating to the deep memory, so as to optimize the support ability for health abnormality detection, diagnosis and treatment.

This disclosure provides dynamic support for abnormality detection model by intelligently managing user's deep memory, such as chronic disease, medication history, and quick memory such as recent blood pressure fluctuations and movement frequency changes, significantly improving the judgment accuracy of health abnormalities. Also, it combines the generative large language model to optimize consultation interaction and provide innovative solutions for chronic disease management and tele-treatment.

This disclosure also relates to a health abnormality detection method, that enables dynamic interaction of multi-layer intelligent memories, comprising following steps:
collecting user's multi-modal physiological signals in real time through wearable devices, preprocessing the collected data and generating time-series data;
performing initial abnormality detection on the generated time-series data, and classifying abnormality detection results;
managing user's quick memory, deep memory and immediate memory, and performing cooperative optimization of health data through memory retrieval and dynamic update; wherein, the quick memory comprises recent health fluctuations, the deep memory stores historical health data of the user, the immediate memory dynamically integrates the quick memory and the deep memory and serves as a dynamic input to provide support for abnormality detection and personalized inquiry;
combining abnormality detection results, user's health memory and medical knowledge base, dynamically generating personalized consultation questions through the generative large language model to interact with the user; wherein, user's feedback is embedded for dynamic updating of memory.

This disclosure also relates to a computer readable storage medium storing a program that enables a computer to execute the health abnormality detection method as described above.

Specifically, according to this disclosure, a health abnormality detection system that enables dynamic interaction of multi-layer intelligent memories, includes:

### Multi-Modal Physiological Data Collection Module

The system collects heterogeneous physiological signals in real time through wearable devices, including but not limited to heart rate, blood pressure, blood oxygen saturation and body temperature. The physiological data is transmitted to the backend of the system at a high frequency.

The data is pre-processed by de-noising and standardizing to form time-series data X={x₁, x₂, . . . , xₙ}, in which xᵢ represents the ith physiological signal.

### Initial Abnormality Detection Module

The initial abnormality detection module performs multi-modal embedding generation and classification, dynamic threshold adjustment and abnormality classification. Specifically, includes the following steps:

(1) performing multi-modal embedding generation and classification, based on the deep sequence model such as Transformer, Bi-LSTM, etc., to accept the input time-series data of the physiological signals, which is converted into a multi-modal health signal embedding E by an encoder. Wherein, E=[e₁, e₂, . . . , eₙ], which is the embedding vector of multi-modal physiological signal.

(2) adopting the adaptive weight mechanism to dynamically adjust the abnormality detection threshold of each signal according to the real-time data, wherein the adjusting formula is:

Tᵢ=T₀+α·Δxᵢ

In which Tᵢ is the adjusted threshold, T₀ is the initial threshold, α is the regulatory factor, and Δxᵢ is the degree to which the signal deviates from the mean.

(3) outputting the abnormality category and emergency severity based on the classifier of the embedding vector E. The results are passed to the memory module for dynamic update and are used as input to the generative interaction and feedback module to optimize the personalized consultation process.

The abnormality category and emergency severity are output based on the classifier of the embedding vector E, wherein the classification probability distribution is given by the following formula:

P(y|E)=f_{c}(W·E+b)

Where, y represents different abnormality category, f_{c} represents the distribution function of the classifier generated probability category, which could use such as Softmax Function. Where, W and b represent the weight matrix and bias vector of the classifier respectively. Based on P(y|E), the system initially outputs the abnormality category and emergency severity score, and generates text explanation, which is passed to the interactive module of generative large language model. P(y|E) is the probability distribution of abnormality category calculated based on W, b and E, representing the possibility of each abnormality category, which is determined by the category y corresponding to the maximum value of probability distribution P(y|E). The initial detection results of the classifier include abnormality category y and emergency severity score s_{E}, which are passed to the memory module for dynamic update and used as input to the generative interaction and feedback module to optimize the personalized consultation process.

### Multi-Layer Intelligent Memory Coordination Module

The multi-layer intelligent memory coordination module can realize the classification of multi-layer memory storage, memory retrieval and auxiliary analysis, and memory dynamic update.

Multilayer memory storage can be classified into deep memory construction, quick memory construction and immediate memory construction. Deep memory contains the user's historical health record, including but not limited to the user's chronic disease record, medication history, sleep regularity and other information. The deep memory embedding vector is denoted as M_{D}, which is stored in the memory database with static and updated properties, and can be used to generate baseline models. Quick memory is used to capture recent health fluctuations, including but not limited to blood pressure changes, exercise frequency, weight changes, etc. Quick memory embedding vector is denoted as M_{Q}. Quick memory is time-sensitive and will be automatically forgotten or reduced to deep memory after a certain period of time. Immediate memory integrates quick memory and deep memory as dynamic input.

The system uses a memory retrieval model to match physiological signal embedding vector E and quick memory embedding vector M_{Q} as well as deep memory embedding vector M_{D}, and uses attention mechanism to calculate correlation:

R=Attention(E,M_{Q},M_{D})

Wherein, R is the retrieved relevant memory which is supplied to the abnormality detection module as input for further optimization of abnormality classification. The Attention Mechanism is a technique widely used in the field of artificial intelligence that aims to solve the problem of information overload by focusing limited computational resources on more important information.

Memory dynamic updating includes quick memory updating and deep memory expansion. The system is based on a sliding time window, and combines with the feedback data to generate a new quick memory embedding vector

$${M}_{Q}^{′}$$

and replace expired information, the formula is:

$${M}_{Q}^{′} = {Update}_{Δ t} ( {M}_{Q} , F )$$

Where, F is the user feedback data, Δt is the sliding time window.

Based on access frequency, information relevance and migration rules, such as the fluctuation of physiological signals lasting longer than a certain time, dynamic transfer the information of quick memory and integrate into deep memory.

### Generative Interaction and Feedback Module

Generative interaction and feedback module can realize dynamic knowledge retrieval, generate personalized inquiry, and feedback processing and memory update. Specifically, the system retrieves domain knowledge K related to abnormality detection results from the medical knowledge base. The retrieval process combines abnormality category y, health signals E and user related health memory data R to ensure the accuracy of knowledge retrieval:

K=TopN(Sim(y+E+R,kᵢ))

Wherein, TopN and Sim are existing functions and could be used directly, kᵢ is the record in the knowledge base.

Combing relevant health memories R, initially detected abnormality category y and emergency severity scores s_{E}, embedding vector E of multi-modal health signals, and health domain knowledge K, the generative large language model dynamically generates a sequence of questions, and the system will interact with the user through natural language questions:

Q=argmax_{q}P(q|E,R,y,s_{E},K)

Where, Q is the generated question sequence, q is the candidate question that may be generated, and argmax_{q} ensures that from all the candidate questions that may be generated, the one with the greatest probability is selected as the final generated question Q. The introduction of health immediate memory R provides the context of health history and helps the generative model generate more personalized query questions. Emergency severity scores s_{E}, abnormality category y, knowledge K, and multi-modal health signals E work together to make inquiry questions more targeted and scientific. For example, high scores s_{E} would trigger a more urgent sequence of questions. Where, argmax_{q} is an existing function and could be used directly.

User feedback could be summarized into embedding vectors by generative models. This feedback supports the system to update the quick memory and decide whether to migrate part of the quick memory to the deep memory, or forget the outdated memory.

This disclosure builds a comprehensive, dynamic and intelligent health abnormality detection system by combining user's physiological data, health memory and generative interaction to provide users with accurate health monitoring and personalized consultation support.

What is claimed is:

1. A health abnormality detection system, that enables dynamic interaction of multi-layer intelligent memories, comprising a multi-modal physiological data collection module, an initial abnormality detection module, a multi-layer intelligent memory coordination module and a generative interaction and feedback module;
wherein, the multi-modal physiological data collection module collects user's multi-modal physiological signals in real time through wearable devices, preprocesses the collected data and generates time-series data, the generated time-series data are provided as input data for subsequent abnormality detection, diagnosis and treatment;
the initial abnormality detection module performs initial abnormality detection on the time-series data generated by the multi-modal physiological data collection module, and classifies abnormality detection results;
the multi-layer intelligent memory coordination module manages the user's quick memory, deep memory and immediate memory, and performs cooperative optimization of health data through memory retrieval and dynamic update; wherein, the quick memory comprises recent health fluctuations, the deep memory stores the historical health data of the user, the immediate memory dynamically integrates the quick memory and the deep memory and serves as a dynamic input to provide support for abnormality detection and personalized inquiry;
the generative interaction and feedback module combines abnormality detection results, user's health memory and medical knowledge base, dynamically generates personalized consultation questions through the generative large language model to interact with the user; simultaneously, the user feedback is embedded and processed for dynamic updating of memory.

2. The health abnormality detection system of claim 1, wherein the initial abnormality detection module performs multi-modal embedding generation and classification, dynamic threshold adjustment and abnormality classification,
in which, the multi-modal embedding generation and classification is based on a deep sequence model to accept the input time-series data of the physiological signals and convert into a multi-modal health signal embedding vector through an encoder;
the dynamic threshold adjustment adopts an adaptive weight mechanism to dynamically adjust an abnormality detection threshold of each signal according to real-time data;
the abnormality classification is based on a classifier of the embedding vector to output abnormality category and emergency degree, the results of which are transmitted to a memory module for dynamic update and used as input for generative interaction and feedback module to optimize a personalized consultation process.

3. The health abnormality detection system of claim 2, wherein the formula for dynamic threshold adjustment is as follows:
Tᵢ=T₀+α·Δxᵢ
where, Tᵢ is an adjusted threshold, T₀ is an initial threshold, α is an regulatory factor, Δxᵢ is a degree to which the signal deviates from the mean.

4. The health abnormality detection system of claim 2, wherein a classifier based on the embedding vector E outputs the abnormality category and the emergency degree, a classification probability distribution of which is generated by the following formula:
P(y|E)=f_{c}(W·E+b)
where, y represents different abnormality category, f_{c} represents the distribution function of the probability classification generated by the classifier, W and b represent the weight matrix and bias vector of the classifier respectively, the system outputs the abnormality category y and emergency severity s_{E} based on P(y|E) and generates text explanation, which is passed to the generative interaction and feedback module.

5. The health abnormality detection system of claim 1, wherein in the multi-layer intelligent memory coordination module, the system uses a memory retrieval model to match physiological signal embedding vector, quick memory embedding vector and deep memory embedding vector, and uses attention mechanism to calculate correlation thereof.

6. The health abnormality detection system of claim 5, wherein the dynamic updating of the memory includes quick memory updating process and deep memory expansion process;
in the quick memory updating process, the system is based on a sliding time window, and combines with a feedback data to generate a new quick memory embedding vector and replace expired information;
in the deep memory expansion process, based on access frequency, information relevance and migration rules, the quick memory information is dynamically transferred and integrated into the deep memory.

7. The health abnormality detection system of claim 1, wherein in the generative interaction and feedback module, the system retrieves domain knowledge related to abnormality detection results from the medical knowledge base, by combing the abnormality category, health signal and user related health memory data.

8. The health abnormality detection system of claim 7, wherein the generative interaction and feedback module combines a relevant health memory, an initially detected abnormality category and an initially detected emergency severity, an embedding vector of multi-modal health signals and a health domain knowledge, and the generative large language model dynamically generates the question sequence, the system interacts with the user through natural language questions.

9. A health abnormality detection method, that enables dynamic interaction of multi-layer intelligent memories, comprising following steps:
collecting user's multi-modal physiological signals in real time through wearable devices, preprocessing the collected data and generating time-series data;
performing initial abnormality detection on the generated time-series data, and classifying abnormality detection results;
managing user's quick memory, deep memory and immediate memory, and performing cooperative optimization of health data through memory retrieval and dynamic update; wherein, the quick memory comprises recent health fluctuations, the deep memory stores historical health data of the user, the immediate memory dynamically integrates the quick memory and the deep memory and serves as a dynamic input to provide support for abnormality detection and personalized inquiry;
combining abnormality detection results, user's health memory and medical knowledge base, dynamically generating personalized consultation questions through the generative large language model to interact with the user; wherein, user's feedback is embedded for dynamic updating of memory.

10. The health abnormality detection method of claim 9, wherein performing initial abnormality detection comprises the following steps:
performing multi-modal embedding generation and classification process, in which, based on the deep sequence model, accepting the input time-series data of physiological signals and converting into a multi-modal health signal embedding vector through an encoder;
performing dynamic threshold adjustment process, in which, adopting an adaptive weight mechanism to dynamically adjust the abnormality detection threshold of each signal according to real-time data; and
performing abnormality classification process, in which, based on a classifier of the embedding vector, outputting abnormality category and emergency degree, the results of which are transmitted to a memory module for dynamic update and used as input for optimizing a personalized consultation.

11. The health abnormality detection method of claim 9, wherein in the process of performing cooperative optimization of health data through memory retrieval and dynamic update, the method uses a memory retrieval model to match physiological signal embedding vector, quick memory embedding vector and deep memory embedding vector, and uses attention mechanism to calculate correlation thereof.

12. The health abnormality detection method of claim 10, wherein the dynamic updating of the memory includes quick memory updating process and deep memory expansion process;
in the quick memory updating process, the method is based on a sliding time window, and combines with feedback data to generate a new quick memory embedding vector and replace expired information;
in the deep memory expansion process, based on access frequency, information relevance and migration rules, the quick memory information is dynamically transferred and integrated into the deep memory.

13. The health abnormality detection method of claim 9, further comprising retrieving domain knowledge related to abnormality detection results from the medical knowledge base, by combing the abnormality category, health signal and user related health memory data.

14. The health abnormality detection method of claim 13, wherein further comprises the following steps:
combing a relevant health memory, an initially detected abnormality category and an initially detected emergency severity, an embedding vector of multi-modal health signal and health domain knowledge;
dynamically generating a question sequence, and
interacting with the user through natural language questions.

15. A computer readable storage medium storing a program that enables a computer to execute the health abnormality detection method according to claim 9.
